(19) United States
(12) Patent Application Publication
(10) Pub. No.: US 2006/0264824 A1
(43) Pub. Date: Nov. 23, 2006

(54) Disposable safety medical syringe assembly and method of manufacture

(76) Inventor: Kyle Young Swisher III, Glenwood, MD (US)

Correspondence Address: Kyle Y. Swisher, III, 14612 Mustang Path, Glenwood, MD, 21738-9517, US

(21) Appl. No.: 11/133,005

(22) Filed: May 20, 2005

(51) Int. Cl. A61M 5/00

(52) U.S. Cl. 604/110; 604/187

(57) ABSTRACT

The invention is a disposable medical safety syringe. The syringe consists of a hollow cylindrical, lens-shaped body; a corresponding plunger rod with a lens-shaped piston head on the anterior of the rod and a flattened base on the posterior of the shaft where thumb pressure is applied during injection; and a sharp, hollow needle secured to the anterior of the syringe body. The piston is manufactured to create a tight hydraulic seal while allowing for free movement of the piston within the hollow syringe body during both fill and injection. The point of connection between the piston and the rod is such to allow the rod to break away from the piston when the rod is twisted within the hollow syringe body as the piston is held securely in place due to its lens shape which corresponds to the lens shape of the hollow syringe outer body.

### CROSS-REFERENCE TO RELATED APPLICATIONS

U.S. Patent Documents

| | | | |
|---|---|---|---|
| D461,243 | S | Aug. 6, 2002 | John J. Niedospil, Jr. |
| D460,820 | S | Jul. 23, 2002 | John J. Niedospil, Jr. |
| D403,761 | | Jan. 5, 1999 | Robert D. Adams |

### STATEMENT REGARDING FEDERALLY SPONSORED RESEARCH OR DEVELOPMENT

No rights to this invention have been reserved under federally-sponsored research and development.

### BACKGROUND OF THE INVENTION

1. Field of the Invention

U.S. Patent Classification 604, Subclass 44 (having body piercing means) & Subclass 110 (having means for preventing reuse of device)

The invention relates to a safety disposable, singly-usable medical syringe wherein the lens shape of the syringe body and plunger head allow for increased viewable surface area while in use thereby reducing the potential for incorrect dosage, and allows for resistance (torque) when twisting the plunger assembly which is detachable.

Current inventions pertaining to safety medical syringes exist however none apply a lens shape to the outer cylindrical body of the device. Previously protected “oval” or “ellipsoidal” shaped syringe bodies reduce the viewable area of the graduated syringe body due to the “rounding” of the cylindrical body as is inherent in the oval or ellipsoidal shape.

Current designs also do not provide for the utility of incorporating a detachable plunger assembly into the design.

Current designs also do not allow for the marking of the syringe body to indicate the orientation of the beveled point of the syringe's needle, the orientation of which is important when injecting serum either subcutaneously or intramuscularly.

2. Description of the Prior Art

| | |
|---|---|
| Adams | D 403,761 |
| Niedospial | D 460,820 |
| Niedospial | D 461,243 |

U.S. Design Patent D403,761, Elliptical Syringe Body, Robert D. Adams, Shamoug, N.J., describes an “elliptical syringe body” wherein the body of the syringe appears to be of an oval shape.

U.S. Design Patent D460,820 S, Ellipsoidal Syringe Barrell, John J. Niedospial, Jr., Burlington, N.J., describes an “ellipsoidal syringe barrel”, wherein the barrel of the syringe is of an oval shape.

U.S. Design Patent D461,243 S, Ellipsoidal Syringe Barrell, John J. Niedospial, Jr., Burlington, N.J., describes a similar ellipsoidal syringe body while providing for ridges on the flanged anterior section of the syringe body.

These patents or known prior uses teach and disclose various types of medical syringes and the like of various designs, but none of them whether taken singly or in combination, disclose the specific details of the invention in such a way as to bear upon the claims of the present invention.

### BRIEF SUMMARY OF THE INVENTION

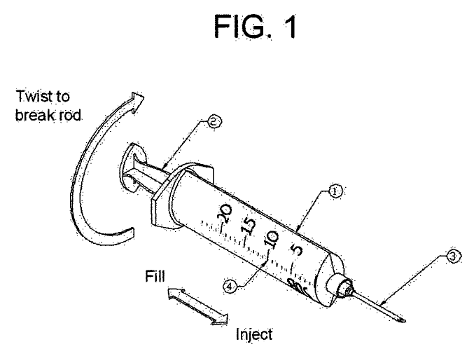

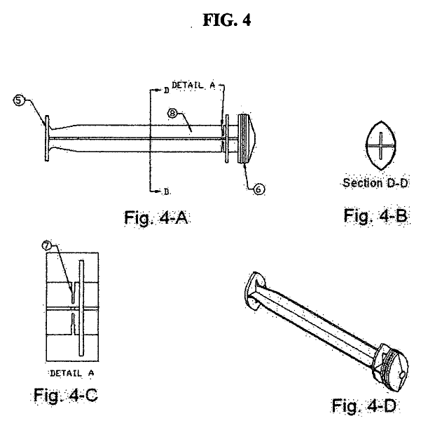

An object, advantage and feature of the invention is to provide a disposable, singly-usable, safety medical syringe comprising a hollow outer syringe chamber (1/FIG. 1) for receiving a piston (2/FIG. 1) that traverses a displaceable extendible path within the outer syringe body comprised of a rod (FIG. 4-A) that has X shape cross section with equal arms, axially coupled to one end of the piston (6/FIG. 4-A) and at the other end to a thumb engaging surface (5/FIG. 4-A) wherein the lens shape of the graduated syringe body allows for several safety advantages.

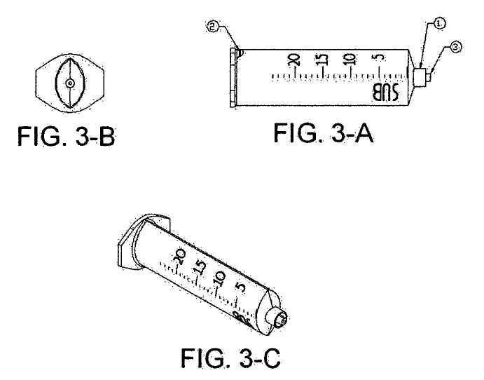

The lens shape of the outer syringe body (FIG. 3) increases the viewable surface of the outer body of the syringe allowing for larger graduated numbering (4/FIG. 1) on the outer surface of the syringe thereby increasing readability and decreasing the potential for improper dosage due to reading errors. Both sides of the syringe outer body are graduated to provide versatility for the user.

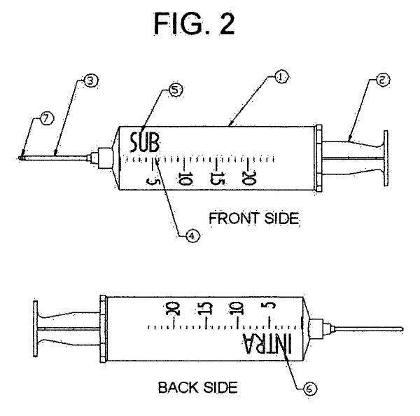

The graduations on both sides of the outer syringe body are identical save for a marking (FIG. 2) on the anterior or posterior end of the body that assists the user in determining the angle of the beveled point (3/FIG. 2) of the needle attached to the anterior end of the syringe body. The orientation of the beveled point of the needle is important to users and its orientation is dictated by a printed marking, either words or diagrams to differentiate either subcutaneous (5/FIG. 2) (under the skin) or intramuscular (6/FIG. 2) (into the muscle) orientation of the attached needle and the corresponding nature of the injection.

The plunger assembly (FIG. 4) is comprised of a single piece of molded plastic having a flattened posterior end (5/FIG. 4-A) for the application of pressure on the plunger, and a molded flange (6/FIG. 4-A) on the anterior of the plunger in a lens shape to create a hydraulic seal within the body of the syringe.

The shape of the plunger shaft is such (FIG. 4-B) to allow the interior wall of the hollow syringe outer shaft to serve as a guide during the plunger's movement.

At the point where the plunger shaft (8/FIG. 4-A) meets the lens shaped guided disc and plunger head (6/FIG. 4-A), the plastic is specifically molded to provide for a break-away spot (7/FIG. 4-C—Detail A) when the plunger shaft is rotated from the posterior end after injection and the torque action causes the separation of the plunger shaft from its lens-shaped head (6/FIG. 4-A) rendering the syringe non-reusable.

3. Brief Description of the Several Views of the Drawing

### DETAILED DESCRIPTION OF THE INVENTION

This invention is a non-reusable hypodermic safety syringe (FIG. 1) consisting of a hollow syringe chamber capable of receiving a piston (FIG. 4) transversing a displaceable distendable path (FIG. 3) in the chamber.

A rod (8—FIG. 4) is axially coupled to one end of the lens-shaped piston (6/FIG. 4), and at the other end to a thumb-engaging surface (5/FIG. 4) and a stop member (2/FIG. 3-A) in the lens-shaped hollow chamber (FIG. 3), is positioned as a proximal end and adjacent to the rod (FIG. 4) for terminating one end of the travel of the displaceable extendible path.

A cap (1/FIG. 3-A) is formed at the distal end of the hollow chamber from the one end and having a tubular coupling (3/FIG. 3-A) for receiving in frictional engagement a syringe needle (3/FIG. 2) and a supporting body for axially centering support of the needle is positioned within the hollow chamber and securely mounted to an inner end of the needle.

A distal end (7/FIG. 2) of the needle (3/FIG. 2) is adapted for injection in a patient for serving to pass fluid (7/FIG. 2), received from the hollow chamber to pass into the patient. The drawing shows the relative displacement of the piston and direction for movement of the piston (2/FIG. 1):
(1) For readying the syringe for intake of serum, by expelling the air in the chamber (FIG. 1) by making a “forward stroke” of the plunger to make a place for the fluid or serum.
(2) To fill the cavity of the syringe with fluid or serum, when the distal end of the needle is inserted in a fluid or serum, by a “reverse stroke”
(3) Dispensing by a second “forward stroke” with normal pressure on the thumb engaging surface and injecting the serum when the distal end of the needle is disposed in a patient,
(4) The separation of the piston rod (FIG. 1), from the piston after removal of the needle from the patient and the twisting of the thumb-engaging device.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be sorted to falling within the scope of the invention.

What I claim as my invention is:

1. A single-use safety medical syringe comprising:
a. a hollow lens-shaped chamber, having a first end and a second end;
b. a lens-shaped piston within said chamber for transversing a displaceable extendible path in said chamber;
c. a rod, whose outer edges form a round cylinder corresponding to the rounded walls of the hollow chamber, having a first end and a second end;
d. a thumb-engaging surface coupled to the second end of said rod;
e. the lens-shaped piston coupled to the first end of the said rod;
f. a stop member formed in the first end of said chamber and adjacent to said rod for terminating one end of the displaceable extendible path;

2. The device of claim 1 wherein said hollow chamber is lens-shaped;

3. The device of claim 1 wherein said piston is lens-shaped;
